(19) United States
(12) Patent Application Publication
(10) Pub. No.: US 2025/0066710 A1
(43) Pub. Date: Feb. 27, 2025

(54) A SINGLE-USE SYSTEM AND METHOD FOR CONTINUOUS HOMOGENIZATION OR LYSIS

(71) Applicant: Athem LLC, Morrisville, NC (US)

(72) Inventor: Sunil Mehta, Morrisville, NC (US)

(21) Appl. No.: 18/726,240

(22) PCT Filed: Mar. 2, 2023
(86) PCT No.: PCT/US2023/063562

§ 371 Date: Jul. 2, 2024

Related U.S. Application Data
(60) Provisional application No. 63/296,984, filed on Jan. 6, 2022.

(51) Int. Cl. C12M 1/00; C12M 1/06; C12M 1/26; C12M 1/34

(52) U.S. Cl. CPC C12M 47/08; C12M 23/28; C12M 27/02; C12M 33/14; C12M 41/00

(57) ABSTRACT

This invention relates to a continuous flow homogenization device and method of using the device for lysis or disruption of cells, tissues, and other biologicals and creating smaller particles and emulsions. The device contains a homogenization chamber in which incoming material continuously enters the chamber via an inlet and the homogenized material continuously flows out via an outlet. The homogenization chamber may include at least one impeller that rotates and/or reciprocates inside the chamber in the presence or absence of a homogenization enhancement media. The homogenization enhancement media may include rigid particles. All product contact surfaces can be sterile and disposable. The complete homogenization process can be executed in a fully automated or manual manner by a controller that manages different inputs and outputs of the system components.

### RELATED APPLICATIONS

This application claims priority from U.S. Provisional Application Ser. No. 63/296,984, filed Jan. 6, 2022, the disclosure of which is incorporated by reference in its entirety.

### FIELD

The present invention is related to methods, devices, and systems that are used for homogenizing organic and inorganic materials.

### BACKGROUND

Lysis or homogenization of cells or tissues is used for many processes to recover valuable products that reside inside cells. For example, recombinant proteins produced by E. coli are contained within the cells. The cells are lysed to release the recombinant proteins for further purification. Similarly, many viruses and viral vectors are produced inside the cells (e.g., Adeno Associated Virus) and require lysis of the cells for their release. With significant shear being produced by homogenizers, they can also be used to reduce size of particles, and to produce emulsions and creams, etc.

There are several distinct methods that can be used for homogenization or lysis of cells from tissue or cell culture. Some of these methods are based on mechanical lysis while others are based on chemical lysis. Although chemical lysis may be used for some small-scale research protocols, it is not a preferred method for manufacturing of biologics as the chemicals must be completely removed from the product after lysis step. Regulatory authorities are limiting the use of detergents that are sometimes used for lysing the cells as the removal of these chemicals is difficult and may require several purification steps. This is one of the primary reasons that commercial discontinuous or continuous homogenization systems are based on mechanical lysis. These systems are cleaned and reused as most of the product contact parts are very expensive and commercially available single-use parts (e.g., tubing, bags) cannot withstand high pressure or high shear that is generated by these homogenization systems. There are some other systems including Dounce homogenizer, ultrasonic homogenizer, and bead mill homogenizer that can be operated in batch mode at small scale but cannot be scaled-up.

Single-use technologies have recently gained significant traction over reusable technologies for biomanufacturing as they eliminate the need for CIP/SIP (clean-in-place/sterilization-in-place) and risk of cross-contamination between batches. They also reduce cycle times between batches and capital investment. Since current homogenization technologies are not single-use, there is a significant need for a single-use homogenization technology.

### SUMMARY

Some embodiments of the present invention are directed to a homogenization device including a chamber including an inlet and an outlet, and at least one impeller in the chamber that is configured to (i) rotate or reciprocate or (ii) rotate and reciprocate. The device is provided sterile before use and is single-use disposable.

In some embodiments, the chamber contains homogenization enhancement media. The homogenization enhancement media may include incompressible or solid particles. The homogenization enhancement media may include incompressible or solid particles sized between 0.1-2000 micron. The homogenization enhancement media may include non-porous particles. The homogenization enhancement media may include porous particles. The homogenization enhancement media may include particles with rough surfaces. The homogenization enhancement media may include particles with smooth surfaces. The homogenization enhancement media may include glass, metal, plastic, ceramic, or any other rigid particles.

In some embodiments, the chamber is made from plastic. In some embodiments, the chamber is made from metal.

In some embodiments, the at least one impeller is connected to a shaft, and the shaft is configured to oscillate.

In some embodiments, the impeller and shaft are made from plastic.

In some embodiments, the impeller and shaft are made from metal.

In some embodiments, all product contact surfaces are disposed after processing a single batch.

In some embodiments, the device is sterilized and packaged prior to its usage.

In some embodiments, the device is configured to homogenize a starting material. The starting material may be cell culture. The starting material may be microbial culture. The starting material may be organic. The starting material may be inorganic.

In some embodiments, the device is configured to lyse cells.

In some embodiments, the device is configured to mix materials.

In some embodiments, the device is configured to reduce the size of starting material.

In some embodiments, the chamber includes a body defining an interior cavity, and the inlet is at a bottom portion of the interior cavity and the outlet is at a top portion of the interior cavity.

In some embodiments, the device includes a filter between the outlet and the interior cavity.

In some embodiments, the device includes a shaft connected to the at least one impeller.

In some embodiments, the at least one impeller is a single blade connected to the shaft.

In some embodiments, a plurality of holes are defined in the single blade impeller.

In some embodiments, the shaft extends from a top of the chamber body.

Some other embodiments of the present invention are directed to a system for homogenization of a starting material. The system includes: a starting material container containing the starting material; a homogenization device including a chamber including an inlet and an outlet, and an impeller that is configured to rotate and/or reciprocate within an interior volume of the chamber; a first tubing line between the starting material container and the inlet of the chamber; a pump (P1) in the first tubing line; a first valve (V1) in the first tubing line; a recirculation container; a second tubing line between the recirculation container and the first tubing line with the second tubing line fluidly connecting the recirculation container and the inlet of the chamber; a second valve (V2) in the second tubing line; a third tubing line between the outlet of the chamber and the starting material container; a third valve (V3) in the third tubing line; a fourth tubing line between the recirculation container and the third tubing line; a fourth valve (V4) in the fourth tubing line; a fifth tubing line between the third tubing line and the first tubing line; a fifth valve (V5) in the fifth tubing line; and a controller configured to direct the first through fifth valves to selectively open and close, to direct the operation of the impeller, and to direct the pump to turn on and turn off.

In some embodiments, the controller is configured to: (a) open the first valve and turn the pump on such that a cycle volume of starting material flows from the starting material container to the inlet and through the homogenization chamber; (b) close the first valve and open the fifth valve such that the cycle volume flows from the outlet to the inlet and through the homogenization chamber a plurality of times; (c) close the fifth valve and open the fourth valve such that the cycle volume is received in the recirculation container; and (d) repeat steps (a) to (c) until the starting material container is depleted of starting material.

In some embodiments, the system further includes a bubble sensor in the first tubing line, and the controller is configured to determine that the starting material container is depleted of starting material based on measurement by the bubble sensor.

In some embodiments, the controller is configured to: (a) open the first valve and turn the pump on such that starting material flows from the starting material container to the inlet and through the homogenization chamber; (b) open the third valve such that the homogenized starting material flows from the outlet to the starting material container; and (c) maintain the first and third valves open and the pump on until desired homogenization is achieved based on measurement by an optical sensor in the first tubing line and/or the third tubing line.

In some embodiments, the controller is configured to: (a) open the first valve, close the second valve, close the third valve, close the fifth valve, open the fourth valve, and turn on the pump such that the starting material flows from the starting material container, through the homogenization device, and to the recirculation container until the starting material container is depleted of starting material; (b) close the first valve, close the fourth valve, and open the second valve and third valve such that the starting material flows from the recirculation container, through the homogenization device, and to the starting material container until the recirculation container is depleted of starting material; and (c) repeat step (a) and/or step (b) a plurality of times until desired homogenization is achieved based on measurement by an optical sensor in the first tubing line and/or the third tubing line.

In some embodiments, the system further includes bubble sensor in the first tubing line, and the controller is configured to determine that the starting material container and the recirculation container is depleted of starting material based on measurement by the bubble sensor.

In some embodiments, the first through fifth tubing lines, the first through fifth valves, a bubble sensor in the first tubing line (where provided), a pressure sensor in the first tubing line (where provided), an optical sensor in the first tubing line (where provided), an optical sensor in the third tubing line (where provided), the homogenization device, the starting material container, the recirculation container, and/or the pump are provided as a kit and are single-use disposable.

Some other embodiments of the present invention are directed to a system for homogenization of a starting material. The system includes: a starting material container containing the starting material; a homogenization device including a chamber including an inlet and an outlet, and an impeller that is configured to rotate and/or reciprocate within an interior volume of the chamber; a first tubing line between the starting material container and the inlet of the chamber; a pump (P1) in the first tubing line; a first valve (V1) in the first tubing line; a second tubing line between the outlet of the chamber and the starting material container; a second valve (V3) in the second tubing line; at least one optical sensor in the first tubing line and/or the second tubing line; and a controller configured to: (a) open the first valve, turn on the pump, and rotate and/or reciprocate the impeller such that starting material flows from the starting material container to the inlet and through the homogenization chamber and is at least partially homogenized; (b) open the second valve such that the at least partially homogenized starting material flows from the outlet to the starting material container; and (c) maintain the first and second valves open and the pump on until desired homogenization is achieved based on measurement by the optical sensor in the first tubing line and/or the second tubing line.

Some other embodiments of the present invention are directed to a method for homogenizing organic and inorganic materials, the method including: flowing starting material from a starting material container through a homogenization device a plurality of times to homogenize the starting material; and collecting the homogenized material in a container.

In some embodiments, the flowing step includes: (a) pumping a cycle volume of the starting material from the starting material container through a first tubing line to an inlet of the homogenization device; (b) closing a first valve (V1) in the first tubing line and opening a second valve (V5) in a second tubing line; (c) flowing the cycle volume from an outlet of the homogenization device through the second and first tubing lines to the inlet and through the homogenization device a plurality of times while operating the homogenization device to homogenize the cycle volume; (d) closing the second valve (V5) and opening a third valve (V4) in a third tubing line so that the homogenized cycle volume enters a recirculation container; and (e) repeating steps (a) through (d) a plurality of times until determining that the starting material container is depleted.

In some embodiments, the method includes using a bubble sensor in the first tubing line for determining that the starting material container is depleted.

In some embodiments, the flowing step includes: (a) pumping starting material from the starting material container through a first tubing line to an inlet of the homogenization device; (b) flowing the starting material through the homogenization device to while operating the homogenization device to at least partially homogenize the starting material; (c) flowing the at least partially homogenized starting material from an outlet of the homogenization device through a second tubing line to the starting material container to mix the at least partially homogenized starting material with the starting material; and (d) repeating steps (a) through (c) until a desired homogenization is achieved.

In some embodiments, the method includes determining when the desired homogenization is achieved using at least one optical sensor (OS1, OS2) in the first tubing line and/or the second tubing line.

In some embodiments, the flowing step includes: (a) opening a first valve (V1) in a first tubing line, opening a second valve (V4) in a second tubing line, closing a third valve (V5) in a third tubing line, closing a fourth valve (V2) in a fourth tubing line, and closing a fifth valve (V3) in a fifth tubing line; (b) pumping the starting material from the starting material container through the first tubing line to an inlet of the homogenization device; (c) flowing the starting material through the homogenization device and operating the homogenization device to at least partially homogenize the starting material; (d) flowing the at least partially homogenized starting material from an outlet of the homogenization device through a fifth tubing line and the second tubing line to a recirculation container until determining that the starting material container is depleted; (e) closing the first valve (V1) in the first tubing line, closing the second valve (V4) in the second tubing line, closing a third valve (V5) in a third tubing line, opening the fourth valve (V2) in the fourth tubing line, and opening the fifth valve (V3) in the fifth tubing line; (f) pumping the at least partially homogenized starting material from the recirculation container through the fourth tubing line to the inlet of the homogenization device; (g) flowing the at least partially homogenized starting material through the homogenization device and operating the homogenization device to further homogenize the starting material; (h) flowing the further homogenized starting material from the outlet of the homogenization device through the fifth tubing line to the starting material container until determining that the recirculation container is depleted; and (i) repeating steps (a) through (h) until a desired homogenization is achieved, while stopping at step (d) to collect homogenized material in the recirculation container, or stopping at step (h) to collect homogenized material in the starting material container.

In some embodiments, the method includes using a bubble sensor in the first tubing line for determining that the starting material container or the recirculation container is depleted.

In some embodiments, the method includes determining when the desired homogenization is achieved using at least one optical sensor (OS1, OS2) in the first tubing line and/or the sixth tubing line.

### BRIEF DESCRIPTION OF THE DRAWINGS

FIG. 1 is a schematic of homogenizer according to some embodiments.

FIGS. 2A-2F illustrate different impeller configurations according to some embodiments.

FIG. 3 is a sectional view illustrating a reciprocating seal according to some embodiments.

FIGS. 4A-4D are charts illustrating experimental results.

FIGS. 5A and 5B are charts illustrating experimental results.

FIG. 6 includes side and front views schematically illustrating external features of a disposable homogenizer chamber according to some embodiments.

FIG. 7 includes rear and side sectional views schematically illustrating internal features of a disposable homogenizer chamber according to some embodiments.

FIG. 8 is a flow schematic of a system according to some embodiments.

### DETAILED DESCRIPTION

Although the embodiments described herein use fluids with cells or particles as a starting material mixture, the device and the methods described herein are also applicable to mixtures containing liquid, solids, gases (e.g., air), and any of their combinations.

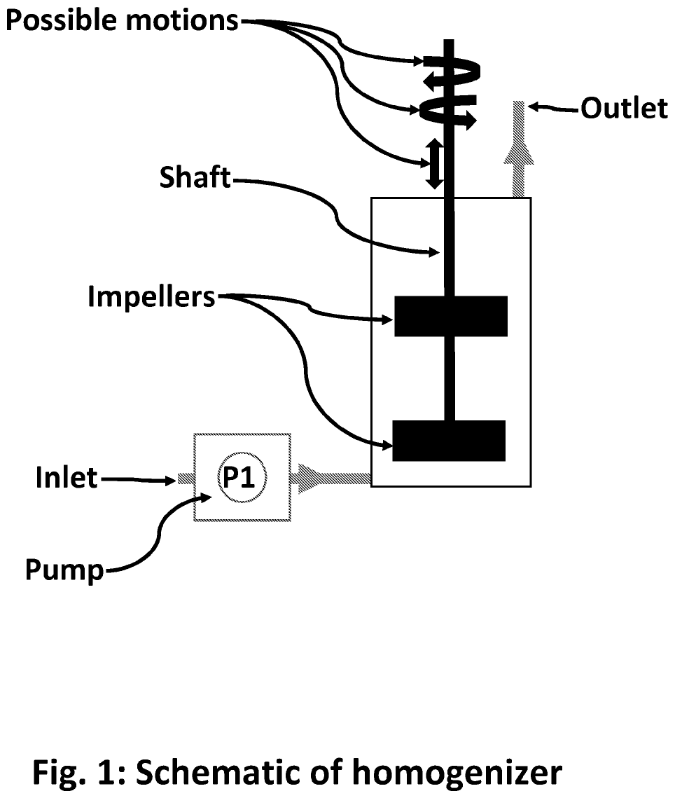

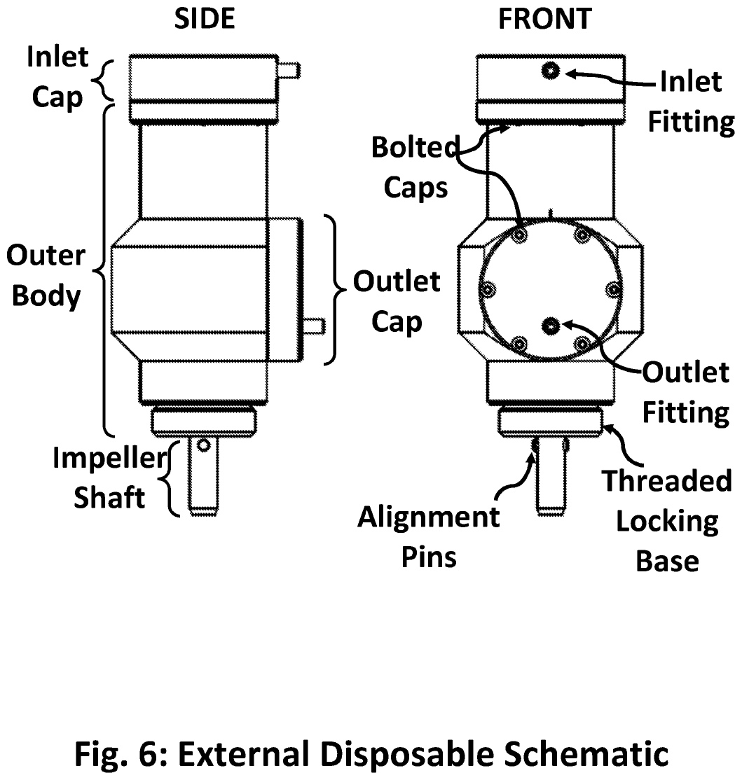

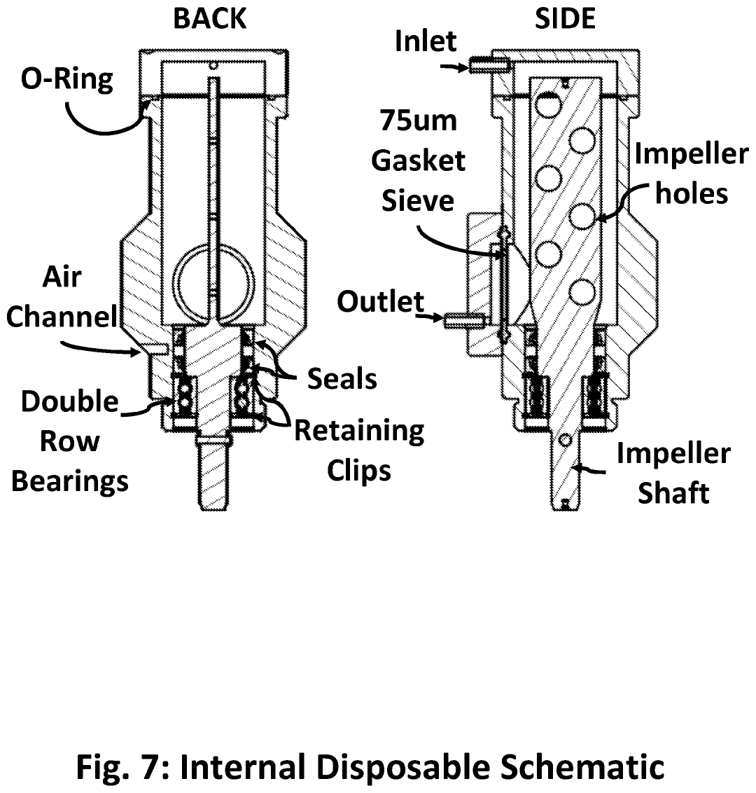

The device and the method described herein uses at least one inlet and at least one outlet (FIG. 1). All product contact surfaces can be disposed after processing a batch. For the biologics manufacturing application, all product contact surfaces are chosen such that they are biocompatible and fit for biologics manufacturing (e.g., C-Flex, silicone, polypropylene, polyethylene, PVC, polycarbonate, 316L stainless steel). The complete homogenization chamber assembly and associated tubing can be pre-sterilized (e.g., by steam, gamma irradiation, ethylene oxide etc.) and packaged prior to its usage. The inlet may be connected to the bottom part of the chamber and the outlet may be connected to the upper part of the chamber (FIG. 1). This configuration allows settling of any lysis or homogenization enhancement media before the lysed product exits the chamber. In addition, this arrangement makes priming simple and removes any bubbles from the outlet that maybe introduced or generated during the process (FIG. 7). A screen filter can be used at the outlet (e.g., FIG. 7 with 75 μm gasket sieve or filter) and/or inlet to contain the homogenization enhancement media in the chamber. The screen filter is installed such that it is pressed against the outer body using a cap that is tightened using screws or bolts (bolted caps in FIG. 6). The pore size in the filters is selected such that it does not allow the escape of homogenization enhancement media particles. The screen filter can be composed of plastic or 316L stainless steel or any other material that is suitable for cGMP manufacturing. The chamber is preferably cylindrical (FIGS. 6 and 7) but other shapes can also be used to improve mixing (e.g., rectangular prism). The chamber may have features (e.g., baffles) to improve mixing and enhance shear. The outer casing of homogenization chamber is composed of plastic but metals such as passivated 316L stainless steel can also be used to manufacture the chamber.

Incompressible beads of various sizes and shapes that are composed of materials with specific gravity greater than 1.0 g/mL can be used as lysis or homogenization enhancement media. The beads are added to the homogenization chamber to improve lysis as the cells passing through the interstitial space between the beads get squeezed, break open, and exude the intracellular material. In one embodiment, borosilicate or soda-lime glass beads are used as the homogenization enhancement media. In another embodiment, stainless steel (e.g., passivated 316L) beads are used. In another embodiment, ceramic beads (e.g., Zirconium oxide beads that are Yttrium stabilized) are used. The size of beads or particles is generally between 0.1-2000 micron and the shape are preferably spherical, spheroidal, rod, cubic, or cuboid but other shapes can also be used. In our results, higher specific gravity media (e.g., ceramic beads with specific gravity of 6.0 g/cm3 in comparison to borosilicated glass beads with specific gravity of 2.7 g/cm3) improved homogenization. Heavier media allowed higher flow rates (10-50%) with similar homogenization results. The beads or particles can have smooth or rough outer surfaces. Non-porous particles are generally preferred but porous particles can also be used. Different types of homogenization enhancement media can be tested with the starting material to determine the most optimal media for that type of starting material.

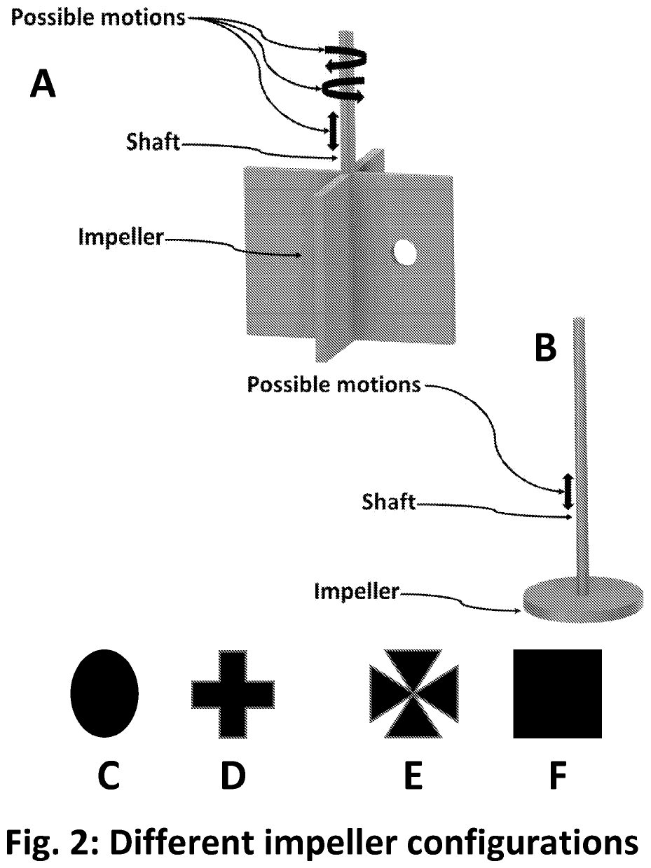

A shaft with a single or multiple impellers are placed inside the chamber (FIGS. 1, 2, and 7). Both shaft and impellers are constructed of rigid material (e.g., 316L stainless steel, polycarbonate etc.). Impeller can be of many different shapes including circular (FIG. 2B), oval (FIG. 2C), polygonal such as rectangular (FIGS. 2F and 7), or cross-shaped (FIGS. 2D and 2E). All these shapes can be with or without holes (FIG. 2A) to improve mixing. In addition to or in lieu of the impeller, a mesh or irregular shape wires (e.g., steel wool) can also be attached to the shaft. The impeller edges can be sharp (e.g., blade surface) to generate high shear at the edge of blade. The sharp edges are generally not required when homogenization enhancement media is used. The plane of the impeller is oriented parallel, perpendicular or at an intermediate angle to the shaft. In one embodiment, the shaft is attached to the impeller at or near the center of impeller. In another embodiment, the shaft is attached to the impeller's side. The shaft and impeller can be manufactured in one piece (FIG. 7). To drive the shaft, alignment pins (FIG. 7) can be used that fit into a slot in the shaft that is connected to the driving motor. The base can have threads (e.g., threaded locking base in FIG. 7) or other mechanisms to lock the outer body. A combination of different shapes of impeller with different motion of shaft can be used. The maximum width of the impeller is smaller than the inside diameter of the chamber and the minimum width is preferably greater than 20 percent of the inside diameter of the chamber.

Rotary or reciprocating motion of the shaft or a combination of rotary and reciprocating motion is used to shear cells with or without the homogenization enhancement media. The shaft can also be oscillated to generate mixing. The motion of the shaft can be driven from the top or the bottom. The reciprocating motion can be rotary (FIG. 2A) or linear (up and down; FIGS. 2A, 2B) or both (FIG. 2A). The speed of the motion can be adjusted by varying the rotational speed (0-50,000 RPM), reciprocating frequency (0-30,000 strokes per minute), or vibration frequency (0-30000 vibrations per second). In our results, the shaft or impeller driven from top or bottom (to provide rotary or reciprocating motion) produced similar results. The stroke length during the reciprocating motion can also be adjusted to improve lysis. The rotary motion can be generated by a motor that is coupled to the shaft either directly or via a set of gears or belt and pulleys. Radial (FIG. 7) or mechanical face seals are used to prevent leaks and maintain a seal between external environment and inside of the chamber. Two seals can be stacked, to create a cavity in which pressure can be maintained by a supply of clean air (air channel in FIG. 7). This air cavity maintains a positive pressure and creates additional air barrier between the seal and the external environment. Generally, the external air pressure is maintained between 2-7 psi. However, depending upon the seal type, higher pressure can also be used. Bearings are installed within the disposable close to the seals and can be held in place by retaining clips (double row bearings in FIG. 7).

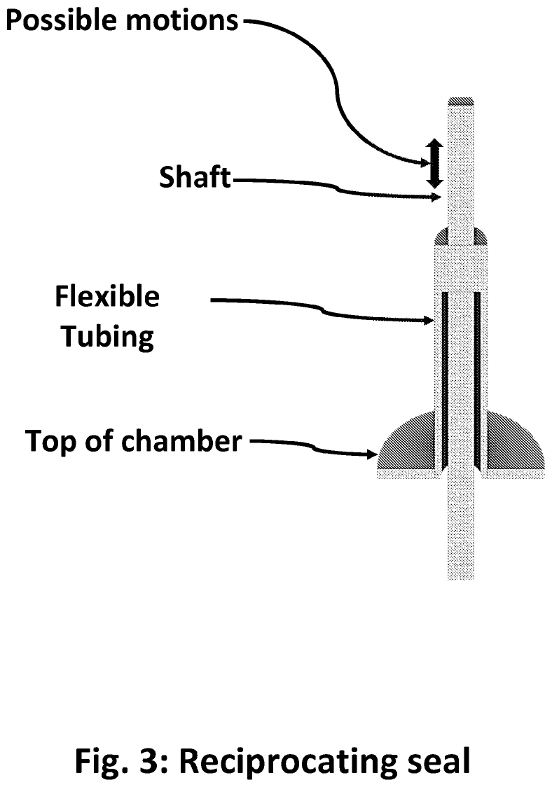

Reciprocating motion to the shaft can be generated by a linear reciprocating motor or a similar mechanism that is used in reciprocating saw. The shaft protruding out of the chamber is clamped or affixed to a flexible tubing or flexible bellow (FIG. 3). The other side of the flexible tubing or flexible bellow is clamped or affixed to the container (FIG. 3). The shaft reciprocates within the flexible tubing or flexible bellow. This configuration creates a sealed system (FIG. 3) and also allows limited rotary (e.g., −180 to +180 degree) with reciprocal motion. If both rotary and reciprocating motions are required, a motor can be mounted to the arm of a reciprocating linear motor to provide both motions simultaneously. Sealing solutions and shaft may not be needed if the impellers are driven via an external magnetic force (e.g., levitation or magnetic drive). Closed system not only maintains sterility but also prevents contamination or exposure of the environment from the potential toxins, pathogens, and other harmful chemicals or agents.

In one embodiment of the application, starting material (e.g., cell culture) is introduced into the homogenization chamber via a pump (e.g., peristaltic pump) through the inlet (FIG. 7). The shaft is rotated and/or reciprocated at a set speed to generate mixing. The pump can be stopped for a short time as soon as the chamber is filled to ensure homogenization of the material from the start. Another way to achieve complete homogenization of the material from the beginning is to start the motion of the shaft prior to introduction of the starting material. If pre-washing is needed, a priming buffer can be introduced in the system prior to introducing the starting material.

The homogenized processed material exits from the outlet (FIG. 7) and is collected in a bag or a vessel. The process continues until all the starting material is pumped to the system. To improve homogenization, the homogenized material can be passed through the system multiple times from collection bag to starting material bag or recirculated. Flexible tubing and pinch valves are controlled via a controller or manually to create the fluid path for recirculation or multiples passes. Once all the starting material has been processed, the residual material in the system can be chased out by a buffer or liquid and the complete disposable fluid path containing the chamber and associated tubing can be sealed and safely discarded.

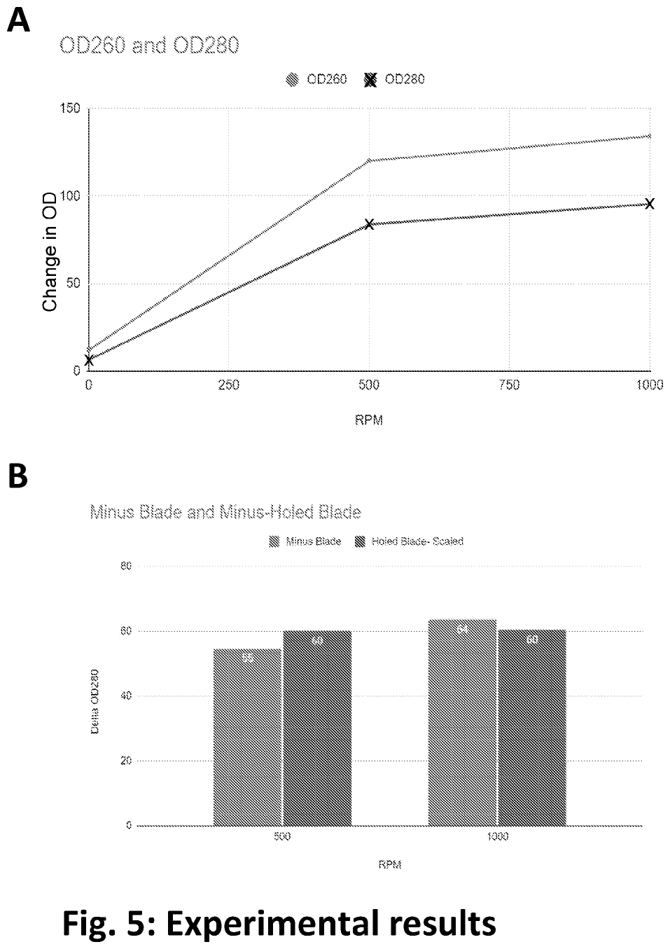

Homogenization of a material can be optimized by either changing the motion of impeller (rpm, oscillation frequency), size and volume of the homogenization enhancement media, shape of the chamber, type of impeller, or flow rate of the starting material. To make the process development easier, the motion of impeller is set to a fixed speed and the inlet flow rate is adjusted to achieve desired homogenization. As the residence time increases with slower flow of transiting liquid mixture, lower flow rates will generally provide higher level of homogenization as compared to higher flow rates. In some cases, higher flow rates may introduce higher level of homogenization due to increased shear. This can be tested by changing the flow rate during process development. Our results show that greater motion of impeller results in higher level of homogenization but may plateau after certain speed (FIGS. 5A and B). Our results indicate that the total volume of the beads is directly proportional to the level of homogenization. This can be used for scaling-up processes. For example, a process at 100 mL/min of flow rate with 125 ml of homogenization enhancement media will provide similar results as a process with 1 L/min flow rate with 1.25 L of homogenization enhancement media. Impellers that generate high shear result in improving homogenization. Overall, increasing shear by different means will result in higher level of homogenization.

The complete system can be manually operated such that flow rate of the pump and speed of impeller is adjusted manually or the system can be fully automated in which the user inputs the parameters into a recipe screen that is linked to a controller, which receives and controls via various digital and analog inputs and outputs. These include pumps (speed, direction, start/stop, feedback etc.), valves (open, close, feedback), occlusion/pressure sensors, optical density and turbidity detectors, bubble sensors, pH, conductivity, temperature sensor, weight load cells, accelerometer, speed, and water leak. Some of the sensors (e.g., occlusion/pressure, accelerometer, water leak, micro-switches) are used to improve safety of the system. For example, if one or more of the tubes are occluded, the pressure rise detected by the occlusion detector or pressure sensor will send a signal to the controller and the system will stop the pumps and open pinch valves to avoid high pressure and potential leaks. This information is provided as analog or digital signals to and from the controller. Commercial automation systems from various vendors such as Siemens, Backhoff, or Allen-Bradley are available as complete solutions that include controller, input/output digital or analog cards, and HMI. Examples are Siemens SIMATIC S7-1200 kit, Delta V automation, or Beckhoff TwinCAT 3 automation platform.

User inputs various process parameters into a process screen that is part of the HMI. This can be turned into a recipe that is executed each time a process is run. Process parameters include flow rates, recirculation times, pH, conductivity, turbidity, optical density, and absorbance values to start or stop a step. A bubble sensor is placed near the inlet to detect emptying of the bioreactor or starting material container.

Experiments were performed to assess the effect of rotation type, speed, impeller shape, and homogenization enhancement media. In some experiments different types of incompressible beads (based on size distribution, shape, or material) were added to the homogenization chamber. The size of the soda-lime glass, borosilicate glass, and ceramic beads ranged from 50-1500 microns.

A shaft with an impeller was placed inside the chamber. A combination of different shapes of impeller with different motion of shaft were used to compare the degree of lysis. Rotary or reciprocating motion of the shaft or a combination of rotary and reciprocating motion were tested to assess the amount of lysis by each combination. The speed of the motion was adjusted by varying the RPM or reciprocating frequency. The stroke length during the reciprocating motion was also adjusted to assess its effect on lysis performance. During the initial screen, the lysis was performed in a batch mode. Based on the lysis performance, selected configurations were used to optimize the flow rate in a continuous mode.

Several different configurations were tested to compare the degree of lysis and homogenization of Saccharomyces cerevisiae. After homogenization, the processed material was centrifuged at 10,000 g for 3 minutes to sediment insoluble content. Absorbance measurement of the centrifuged supernatant at OD280 (to quantify relative intracellular protein release) and OD260 (to quantify relative intracellular DNA and RNA release) provided an effective way to quantify lysis. The amount of lysis was calculated as percent increase from the basal levels. In addition, the lysis was verified qualitatively by viewing the culture under a microscope.

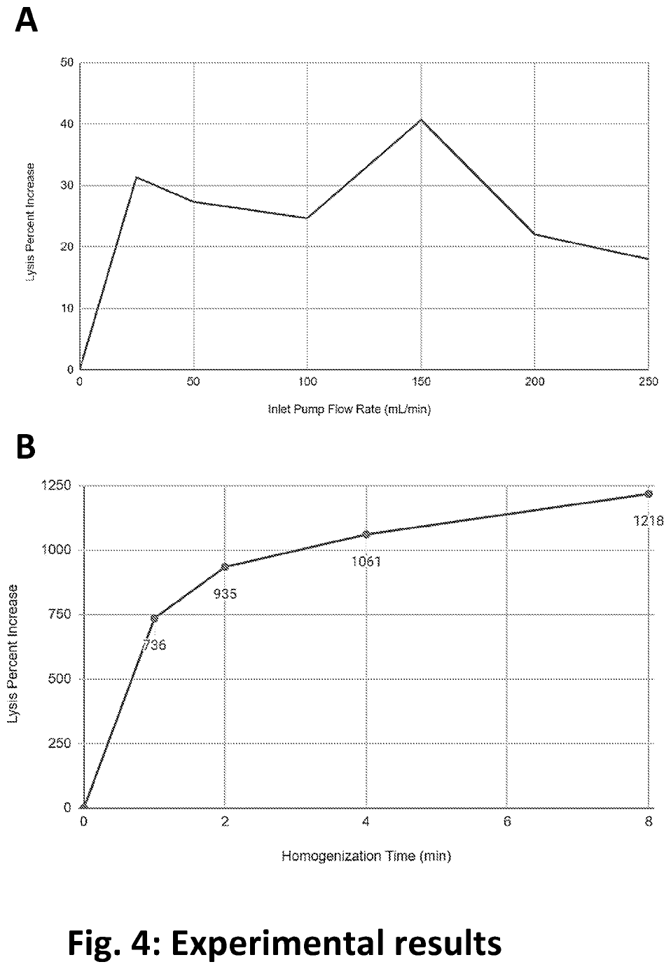

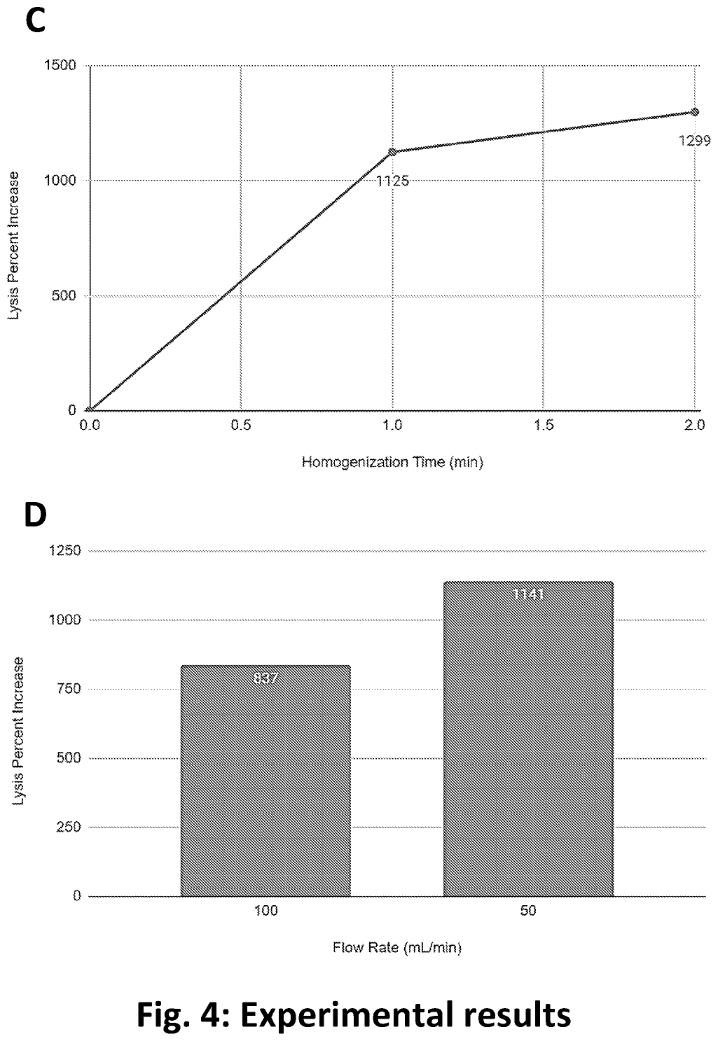

Results from FIG. 4A were obtained from a configuration of a shaft rotating at 3500 RPM with 24 impellers (as shown in FIG. 2E) equidistant placed and mounted horizontally to the vertical shaft. The shape of the chamber was cylindrical with inlet in the bottom and outlet on the top. These results show minimal lysis (less than 20%) at all the tested flow rates in the absence of homogenization enhancement media.

In another experiment (FIG. 4B), a shaft mounted with paddle mixer (FIG. 2F, mounted parallel to vertical shaft) was used in a cylindrical chamber and rotated at 200 RPM in the presence of 500 micron soda-lime glass beads. These results show 10-fold increase in the amount of protein released and almost complete lysis (as observed under the microscope) in less than eight minutes. For continuous flow experiments, this shaft was mounted to a paddle mixer with and without holes (FIGS. 6 and 7). These impellers were used in a cylindrical chamber and rotated at 500 RPM or 1000 RPM in the presence of 500 micron soda-lime glass beads. After priming, the flow rate was continuously maintained at 50 mL/min. The results show that there is not much increase in lysis (as indicated by OD280 and OD260) with increase in RPM beyond 500 (FIG. 5A). Paddle mixer with and without holes were also tested. Our results from OD280 measurements (FIG. 5B) indicate that there was not significant homogenization difference between the two configurations at two different RPMs (500 and 1000).

To test the reciprocating shaft mixing, a shaft with FIG. 2B configuration was placed in a cylindrical chamber and reciprocated at 2400 strokes per minute in the presence of 500 micron soda-lime glass beads. These results (FIG. 4C) show almost complete lysis in almost one minute. The complete system was cleaned, and the starting material was continuously fed into the homogenization chamber via a peristaltic pump set at 50 and 100 mL/min flow rate. The results (FIG. 4D) show greater than 10-fold lysis at 50 mL/min flow rate. When the homogenized samples were observed under the microscope, most of the cells appeared lysed.

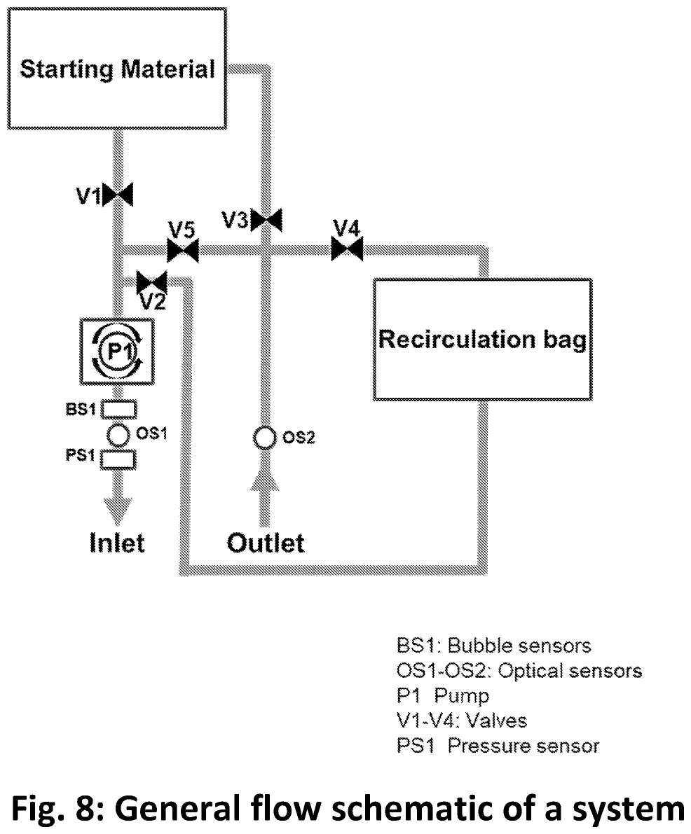

FIG. 8 shows a general flow schematic of a system. The flow rates, impeller speed or frequency, and other critical parameters are entered through the HMI screen by the user. V1-V5 are pinch valves and are closed at the start of a process. The homogenization chamber is completely assembled along with the homogenization media. The pressure or occlusion sensor PS1 is used to monitor pressure in the system and if the pressure exceeds the safety pressure limit (set in the HMI), the pump stops and all valves go to the closed state. At the beginning of the process, the starting material (e.g., cell culture, microbial culture, mixture of liquids, mixture of liquid and solid, mixture of gas and solid, mixture of gas and liquid etc.) from a bag or vessel enters the homogenization chamber via flowing through a tubing (through pump P1) that goes through the bubble sensor BS1, open pinch valve V1, optical sensor OS1, and pressure sensor PS1. The impeller in the homogenization chamber starts once the liquid enters the system (when the bubble sensor BS1 detects liquid plus the time it takes to for the starting material to travel the set length of tubing at the set flow rate). The homogenized material goes out through the outlet tubing via an optical sensor OS2 and open pinch valve V4 into the recirculation bag. The homogenization efficiency can be monitored by the optical sensors (e.g., measuring the OD280 for change in the protein content or OD260 to measure the change in DNA content). This process continues, until the bubble sensor BS1 detects steady stream of air as the starting material bag runs out of the starting material. At this point, homogenized material in the recirculation bag can be used for the subsequent process. A buffer can be used to prime the system in the beginning of the process and chase out remaining material inside the homogenization chamber at the end of process.

There may be a plurality of tubing lines in the system. For example, there may be a first tubing line between the starting material container and the inlet of the homogenization chamber, a second tubing line between the recirculation container and the inlet of the homogenization chamber, a third tubing line between the outlet of the homogenization chamber and the starting material chamber, a fourth tubing line between the recirculation container and the third tubing line, and/or a fifth tubing line between the third tubing line and the first tubing line. As used herein, the term “tubing line” generally means a fluid path and may include one or more (flexible) tubes, fittings, manifolds, and the like. As seen in FIG. 8, there may be components such as pumps, sensors, and valves in the various tubing lines.

To improve homogenization, three strategies can be used in addition to increasing the impeller speed or reducing the flow rate of the pump P1 (inlet flow rate). In all cases, all valves are closed at the beginning of the process.

In the first strategy, the starting material flows or passes through the homogenization chamber multiple times before sending it to the recirculation bag. The following steps are conducted.

Step 1: A set volume (cycle volume) of the starting material is pulled through open valve V1 by the pump P1 and is pushed into the inlet of the homogenization chamber. The homogenized material coming out of the chamber via the outlet goes through open valve V5 to the inlet again (with valve V1 closed) until desired cycles have been completed. Valves V2, V3, and V4 are closed during this step.

Step 2: At that stage, valve V1 opens, valve V5 closes, and valve V4 opens so that the material equal to the cycle volume goes to the recirculation bag.

Step 3: Go to Step 1 and this process continues, until the bubble sensor BS1 detects steady stream of air, which shows end of the starting material. At this point homogenized material in the recirculation bag can be used for the subsequent process.

In the second strategy, the homogenized material is sent back to the starting material bag continuously until the starting material is homogenized to the desired extent. In this case, the final material is collected in the same bag or vessel as the starting material. The starting material is pulled through open valve V1 by the pump P1 and pushed into the inlet of the homogenization chamber. The material coming out of the chamber via the outlet goes through open valve V3 to the starting material and mixes with it. The process is stopped when the desired homogenization is achieved. At this point homogenized material in the original starting material bag can be used for the subsequent process. If present, the valves V2 and V4 are closed in this strategy. The valves V2 and V4 and the recirculation container (and associated tubing lines) may be omitted from the system for this strategy.

In the third strategy, the following steps are performed.

Step 1: Homogenized material is collected in the recirculation bag as per the sequence described above where no recirculation is performed.

Step 2: Once the starting material is completely out of the original bag, the homogenized material collected in the recirculation bag is fed back to the homogenization chamber by pump P1 via open valves V2 and V3 and closed valves V1, V4, and V5. This process continues until the recirculation bag becomes empty (steady air in the bubble sensor BS1).

Step 3: Step 1 is initiated for a desired number of cycles. After that process is stopped either after Step 1 and the final processed material is collected from the recirculation bag or Step 2 and the final processed material is collected from the starting material bag.

The recirculation processes as described above can also be combined to improve homogenization efficiency.

The present invention has been described herein with reference to the accompanying drawings, in which illustrative embodiments of the invention are shown. In the drawings, the relative sizes of regions or features may be exaggerated for clarity. This invention may, however, be embodied in many different forms and should not be construed as limited to the embodiments set forth herein; rather, these embodiments are provided so that this disclosure will be thorough and complete, and will fully convey the scope of the invention to those skilled in the art.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention.

Spatially relative terms, such as “beneath”, “below”, “lower”, “above”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” can encompass both an orientation of above and below. The device may be otherwise oriented (rotated 90° or at other orientations) and the spatially relative descriptors used herein interpreted accordingly.

As used herein, the singular forms “a”, “an” and “the” are intended to include the plural forms as well, unless expressly stated otherwise. It will be further understood that the terms “includes,” “comprises,” “including” and/or “comprising,” when used in this specification, specify the presence of stated features, integers, steps, operations, elements, and/or components, but do not preclude the presence or addition of one or more other features, integers, steps, operations, elements, components, and/or groups thereof. It will be understood that when an element is referred to as being “connected” or “coupled” to another element, it can be directly connected or coupled to the other element or intervening elements may be present. As used herein, the term “and/or” includes any and all combinations of one or more of the associated listed items. When the term “about” or “substantially equal to” is used in the specification the intended meaning is that the value is plus or minus 5% of the specified value.

It is noted that any one or more aspects or features described with respect to one embodiment may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail in the specification set forth herein.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The foregoing is illustrative of the present invention and is not to be construed as limiting thereof. Although a few example embodiments of this invention have been described, those skilled in the art will readily appreciate that many modifications are possible in the example embodiments without materially departing from the teachings and advantages of this invention. Accordingly, all such modifications are intended to be included within the scope of this invention as defined in the claims. The invention is defined by the following claims, with equivalents of the claims to be included therein.

1. A homogenization device comprising:
a chamber comprising an inlet and an outlet; and
at least one impeller in the chamber that is configured to (i) rotate or reciprocate or (ii) rotate and reciprocate,
wherein the device is provided sterile before use and is single-use disposable.

2. The device of claim 1 wherein the chamber contains homogenization enhancement media.

3. The device of claim 2 wherein the homogenization enhancement media comprises: incompressible or solid particles; incompressible or solid particles sized between 0.1-2000 micron; non-porous particles; porous particles; particles with rough surfaces; particles with smooth surfaces; and/or glass or metal particles.

4-9. (canceled)

10. The device of claim 1 wherein the chamber is made from plastic or metal.

11. (canceled)

12. The device of claim 1 wherein the at least one impeller is connected to a shaft, and wherein the shaft is configured to oscillate.

13. The device of claim 12 wherein the impeller and shaft are made from plastic or metal.

14. (canceled)

15. The device of claim 1 wherein all product contact surfaces are disposed after processing a single batch.

16. The device of claim 1 wherein the device is sterilized and packaged prior to its usage.

17-24. (canceled)

25. The device of claim 1 wherein the chamber comprises a body defining an interior cavity, and wherein the inlet is at a bottom portion of the interior cavity and the outlet is at a top portion of the interior cavity.

26. The device of claim 25 further comprising a filter between the outlet and the interior cavity.

27. The device of claim 25 further comprising a shaft connected to the at least one impeller.

28. The device of claim 27 wherein the at least one impeller is a single blade connected to the shaft, optionally with a plurality of holes defined in the single blade impeller.

29. (canceled)

30. The device of claim 27 wherein the shaft extends from a top of the chamber body.

31. A system for homogenization of a starting material, the system comprising:
a starting material container containing the starting material;
a homogenization device comprising: a chamber including an inlet and an outlet; and an impeller that is configured to rotate and/or reciprocate within an interior volume of the chamber;
a first tubing line between the starting material container and the inlet of the chamber;
a pump (P1) in the first tubing line;
a first valve (V1) in the first tubing line;
a recirculation container;
a second tubing line between the recirculation container and the first tubing line with the second tubing line fluidly connecting the recirculation container and the inlet of the chamber;
a second valve (V2) in the second tubing line;
a third tubing line between the outlet of the chamber and the starting material container;
a third valve (V3) in the third tubing line;
a fourth tubing line between the recirculation container and the third tubing line;
a fourth valve (V4) in the fourth tubing line;
a fifth tubing line between the third tubing line and the first tubing line;
a fifth valve (V5) in the fifth tubing line; and
a controller configured to direct the first through fifth valves to selectively open and close, to direct the operation of the impeller, and to direct the pump to turn on and turn off.

32. The system of claim 31 wherein the controller is configured to:
(a) open the first valve and turn the pump on such that a cycle volume of starting material flows from the starting material container to the inlet and through the homogenization chamber;
(b) close the first valve and open the fifth valve such that the cycle volume flows from the outlet to the inlet and through the homogenization chamber a plurality of times;
(c) close the fifth valve and open the fourth valve such that the cycle volume is received in the recirculation container; and
(d) repeat steps (a) to (c) until the starting material container is depleted of starting material.

33. The system of claim 32 further comprising a bubble sensor in the first tubing line, wherein the controller is configured to determine that the starting material container is depleted of starting material based on measurement by the bubble sensor.

34. The system of claim 31 wherein the controller is configured to:
(a) open the first valve and turn the pump on such that starting material flows from the starting material container to the inlet and through the homogenization chamber;
(b) open the third valve such that the homogenized starting material flows from the outlet to the starting material container; and
(c) maintain the first and third valves open and the pump on until desired homogenization is achieved based on measurement by an optical sensor in the first tubing line and/or the third tubing line.

35. The system of claim 31 wherein the controller is configured to:
(a) open the first valve, close the second valve, close the third valve, close the fifth valve, open the fourth valve, and turn on the pump such that the starting material flows from the starting material container, through the homogenization device, and to the recirculation container until the starting material container is depleted of starting material;
(b) close the first valve, close the fourth valve, and open the second valve and the third valve such that the starting material flows from the recirculation container, through the homogenization device, and to the starting material container until the recirculation container is depleted of starting material; and
(c) repeat step (a) and/or step (b) a plurality of times until desired homogenization is achieved based on measurement by an optical sensor in the first tubing line and/or the third tubing line.

36. The system of claim 35 further comprising a bubble sensor in the first tubing line, wherein the controller is configured to determine that the starting material container and the recirculation container is depleted of starting material based on measurement by the bubble sensor.

37. The system of claim 31 wherein the first through fifth tubing lines, the first through fifth valves, a bubble sensor in the first tubing line (where provided), a pressure sensor in the first tubing line (where provided), an optical sensor in the first tubing line (where provided), an optical sensor in the third tubing line (where provided), the homogenization device, the starting material container, the recirculation container, and/or the pump are provided as a kit and are single-use disposable.

38. A system for homogenization of a starting material, the system comprising:
a starting material container containing the starting material;
a homogenization device comprising: a chamber including an inlet and an outlet; and an impeller that is configured to rotate and/or reciprocate within an interior volume of the chamber;
a first tubing line between the starting material container and the inlet of the chamber;
a pump (P1) in the first tubing line;
a first valve (V1) in the first tubing line;
a second tubing line between the outlet of the chamber and the starting material container;
a second valve (V3) in the second tubing line;
at least one optical sensor in the first tubing line and/or the second tubing line; and
a controller configured to:
(a) open the first valve, turn on the pump, and rotate and/or reciprocate the impeller such that starting material flows from the starting material container to the inlet and through the homogenization chamber and is at least partially homogenized;
(b) open the second valve such that the at least partially homogenized starting material flows from the outlet to the starting material container; and
(c) maintain the first and second valves open and the pump on until desired homogenization is achieved based on measurement by the optical sensor in the first tubing line and/or the second tubing line.

39. A method for homogenizing organic and inorganic materials, the method comprising:
flowing starting material from a starting material container through a homogenization device a plurality of times to homogenize the starting material; and
collecting the homogenized material in a container.

40. The method of claim 39 wherein the flowing step comprises:
(a) pumping a cycle volume of the starting material from the starting material container through a first tubing line to an inlet of the homogenization device;
(b) closing a first valve (V1) in the first tubing line and opening a second valve (V5) in a second tubing line;
(c) flowing the cycle volume from an outlet of the homogenization device through the second and first tubing lines to the inlet and through the homogenization device a plurality of times while operating the homogenization device to homogenize the cycle volume;
(d) closing the second valve (V5) and opening a third valve (V4) in a third tubing line so that the homogenized cycle volume enters a recirculation container; and
(e) repeating steps (a) through (d) a plurality of times until determining that the starting material container is depleted.

41. The method of claim 40 comprising using a bubble sensor in the first tubing line for determining that the starting material container is depleted.

42. The method of claim 39 wherein the flowing step comprises:
(a) pumping starting material from the starting material container through a first tubing line to an inlet of the homogenization device;
(b) flowing the starting material through the homogenization device to while operating the homogenization device to at least partially homogenize the starting material;
(c) flowing the at least partially homogenized starting material from an outlet of the homogenization device through a second tubing line to the starting material container to mix the at least partially homogenized starting material with the starting material; and
(d) repeating steps (a) through (c) until a desired homogenization is achieved.

43. The method of claim 42 further comprising determining when the desired homogenization is achieved using at least one optical sensor (OS1, OS2) in the first tubing line and/or the second tubing line.

44. The method of claim 39 wherein the flowing step comprises:
(a) opening a first valve (V1) in a first tubing line, opening a second valve (V4) in a second tubing line, closing a third valve (V5) in a third tubing line, closing a fourth valve (V2) in a fourth tubing line, and closing a fifth valve (V3) in a fifth tubing line;
(b) pumping the starting material from the starting material container through the first tubing line to an inlet of the homogenization device;
(c) flowing the starting material through the homogenization device and operating the homogenization device to at least partially homogenize the starting material;
(d) flowing the at least partially homogenized starting material from an outlet of the homogenization device through a sixth tubing line and the second tubing line to a recirculation container until determining that the starting material container is depleted;
(e) closing the first valve (V1) in the first tubing line, closing the second valve (V4) in the second tubing line, closing a third valve (V5) in a third tubing line, opening the fourth valve (V2) in the fourth tubing line, and opening the fifth valve (V3) in the fifth tubing line;
(f) pumping the at least partially homogenized starting material from the recirculation container through the fourth tubing line to the inlet of the homogenization device;
(g) flowing the at least partially homogenized starting material through the homogenization device and operating the homogenization device to further homogenize the starting material;
(h) flowing the further homogenized starting material from the outlet of the homogenization device through the sixth tubing line and the fifth tubing line to the starting material container until determining that the recirculation container is depleted; and
(i) repeating steps (a) through (h) until a desired homogenization is achieved, while stopping at step (d) to collect homogenized material in the recirculation container, or stopping at step (h) to collect homogenized material in the starting material container.

45. The method of claim 44 comprising using a bubble sensor in the first tubing line for determining that the starting material container or the recirculation container is depleted.

46. The method of claim 44 further comprising determining when the desired homogenization is achieved using at least one optical sensor (OS1, OS2) in the first tubing line and/or the sixth tubing line.
